(19) United States
(12) Patent Application Publication
(10) Pub. No.: US 2021/0196591 A1
(43) Pub. Date: Jul. 1, 2021

(54) NATURE-BASED COMPOSITIONS, DISPENSING SYSTEMS AND METHODS FOR PROVIDING SENSORIAL AND AESTHETIC BENEFITS TO ACNE PRONE SKIN

(71) Applicant: L'OREAL, Paris (FR)

(72) Inventors: Stephanie Anne Venture Morris, Montclair, NJ (US); Ryuji Hara, Westfield, NJ (US)

(73) Assignee: L'OREAL, Paris (FR)

(21) Appl. No.: 16/727,908

(22) Filed: Dec. 26, 2019

(51) Int. Cl. A61K 8/368; A61K 8/73; A61K 8/99; A61K 8/34; A61K 8/96; A45D 34/04

(52) U.S. Cl. CPC A61K 8/368; A61K 8/737; A61K 8/99; A61K 8/34; A45D 2200/1036; A61K 8/965; A45D 34/04; A45D 2200/1018; A61K 8/731

(57) ABSTRACT

A cosmetic composition includes at least one beta hydroxy acid selected from -n-octanoylsalicylic acid, salicylate, sodium salicylate, willow extract, beta hydroxybutanoic acid, tropic acid, and trethocanic acid present in a hydroalcoholic gel dispersion that includes at least one C2-C5 alcohol, (ii a blend of at least two powders selected from at least one inorganic powder and at least one nature-based organic powder, and (iii) at least one nature-based polymer that excludes synthetic organic polymers. The cosmetic composition may be dispensed using a porous applicator and is applied as an on-the-go skin refresher that is especially good for acne-prone skin. The cosmetic composition can be used throughout the day to control shine and minimize skin imperfections and discourage acne and is suitable for use alone or over or under make up.

### FIELD OF THE INVENTION

The present disclosure is directed to nature-based skin care compositions cosmetic dispensing systems, and methods that provide sensorial and aesthetic benefits to acne prone skin. The benefits include shine and acne control, in a light and refreshing cooling gel that can be applied alone, under or over makeup.

### BACKGROUND OF THE INVENTION

Prior art compositions that have a low pH and high salt content, such as hydroxy acid-containing compositions, typically employ one or more of surfactants and high amounts of polymeric thickeners to maintain stability and consistency while maintaining the solubility of the acid. Inclusion of emulsifying surfactants and high amounts of polymeric thickeners can be associated with tackiness, unwanted whitening upon application, and with skin drying and irritation and overall loss of skin hydration. In addition to the forgoing, compositions that employ surfactants and high amounts of polymeric thickeners do not provide a fresh skin feel, do not diminish oiliness or minimize skin imperfections, and cannot be conveniently applied over or under makeup.

Accordingly, there is a need for a composition that overcomes the shortcomings of the prior art and provides benefits that include the ability to incorporate amounts of soluble hydroxy acids, for example salicylic acid, effective for treating acne, in a stable gel dispersion of oil absorbing powders, without emulsifying surfactants and without high content of polymers.

### BRIEF SUMMARY OF THE INVENTION

In accordance with the various embodiments, provided are compositions, cosmetic dispensing systems, and methods that provide sensorial and aesthetic benefits to acne prone skin. The benefits include shine and acne control, in a light and refreshing cooling gel that can be applied alone, under or over makeup.

In various embodiments, the disclosure provides a cosmetic composition that includes at least one beta hydroxy acid selected from -n-octanoylsalicylic acid, salicylate, sodium salicylate, willow extract, beta hydroxybutanoic acid, tropic acid, and trethocanic acid. The at least one beta hydroxy acid is present in a hydroalcoholic gel dispersion. The hydroalcoholic gel dispersion includes (i) at least one C2-C5 alcohol, (ii) a blend of at least two powders selected from at least one inorganic powder and at least one nature-based organic powder, and (iii) at least one nature-based polymer that excludes synthetic organic polymers

In some embodiments, the at least one beta hydroxy acid is salicylic acid. In some embodiments, the at least one beta hydroxy acid comprises salicylic acid present in the composition in the range from about 0.2% to about 2%, by weight, based on the total weight of the composition.

In some embodiments, the at least one C2-C5 alcohol is selected from ethanol, propanol, butanol, pentanol, isopropanol, isobutanol and isopentanol. In some embodiments, the at least one C2-C5 alcohol comprises denatured alcohol present in the composition in the range from about 5% to about 20%, by weight, based on the total weight of the composition

In some embodiments, the pH of the cosmetic composition is in the range from about 3.9 to about 4.5.

In some embodiments, the blend of powders comprises perlite, and one or more of cellulose, microcrystalline cellulose and kaolin. In some embodiments, the at least one inorganic powder is present in the cosmetic composition in an amount that ranges from about 0.5% to about 1% by weight, and each one of the at least one organic powder is present in the cosmetic composition in an amount that ranges from about 0.25% to about 2% by weight, by weight based on the weight of the composition.

In some embodiments, the at least one nature-based polymer is not electrolyte sensitive or salt intolerant. In some embodiments, the at least one nature-based polymer comprises a blend of two or more polymers selected from acacia senegal gum, Caesalpinia spinosa gum, agar, carob gum, carrageenan, carrageenan (lambda, iota kappa), cellulose gum, corn starch modified, dehydroxanthan gum, gellan gum, guar gum, guar hydroxypropyltrimonium chloride, gum acacia, gum arabic, gum ghatti, gum karaya, gum tragacanth, hydroxypropyl guar, hydroxypropyl methyl cellulose, lecithin, microcrystalline cellulose, pectin, potato starch modified, pullulan, sclerotium gum, sodium carboxymethyl starch, sorbitol/sebacic acid copolymer, xanthan gum, and mixtures thereof. In some embodiments, the at least one nature based polymer includes one of a blend of xanthan gum and sclerotium gum or hydroxypropyl guar. In some embodiments, the at least one nature based polymer includes a blend of xanthan gum and sclerotium gum and hydroxypropyl guar. In some embodiments, the at least one nature-based polymer is present in an amount that ranges from about 0.2% to about 0.6% by weight.

In some embodiments, the cosmetic composition excludes synthetic polymers selected from Ammonium Acryloyldimethyltaurate/Steareth-25 Methacrylate Crosspolymer, and one of polyacrylate crosspolymer 6 and an acrylates/C10-30 Alkyl acrylate crosspolymer. In some embodiments, the cosmetic composition excludes polymers selected from dilinoleic acid/butanediol copolymer, dimer dilinoleyl dimer dilinoleate, algin, oxidized starch acetate, cyclodextrin, hydroxypropyl guar hydroxypropyltrimonium chloride, sodium dextran sulfate, polyhydroxylstearic acid, microcrystalline cellulose (and) cellulose gum, biosaccharide gum-1 (and) sodium levulinate (and) glyceryl caprylate (and) sodium anisate, and mannitol (and) cellulose (and) hydroxypropyl methyl cellulose.

In some embodiments, the cosmetic composition has a gel crossover point ranging from about 35% to about 85% strain. In some embodiments, the cosmetic composition has a gel crossover point which allows it to suspend the at least one powder and pass through a porous applicator with shear.

In some particular embodiments, the cosmetic composition includes a hydroalcoholic gel dispersion, comprising a blend of (i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers, (ii) at least one C2-C5 alcohol, (iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and (iv) a blend of at least two powders, at least one powder selected from inorganic powders and organic powders, and at least one nature-based organic powder, wherein the at least one beta hydroxy acid is dissolved in the hydroalcoholic gel dispersion and the blend of at least two powders is dispersed in the hydroalcoholic gel dispersion.

In some particular embodiments, the cosmetic composition includes: (a) salicylic acid present in an amount in the range from about 0.2% to about 2%, and an (b) aqueous hydroalcoholic gel dispersion, comprising: (i) denatured alcohol in an amount that is about 20% by weight, (ii) perlite present in the cosmetic composition in an amount that ranges from about 0.5% to about 1% by weight, microcrystalline cellulose present in the cosmetic composition in an amount that ranges from about 1.4% to about 2% by weight, and one or more of cellulose and kaolin, each present in the cosmetic composition in an amount that ranges from about 0.25% to about 1% by weight, and (iii) at least one of a blend of xanthan gum and sclerotium gum and hydroxypropyl guar, the blend of xanthan gum and sclerotium gum, when present, is present in an amount that ranges from about 0.25% to about 0.75% by weight, and the hydroxypropyl guar, when present, is present in an amount that ranges from about 0.1% to about 0.4% by weight.

In some embodiments, the invention also relates to a cosmetic dispensing system for addressing shine and acne that includes a cosmetic composition and a dispenser.

In various embodiments, the disclosure provides a cosmetic dispensing system that includes a cosmetic composition that has a gel crossover point suitable for dispensing through a porous applicator, the cosmetic composition including (a) at least one beta hydroxy acid selected from -n-octanoylsalicylic acid, salicylate, sodium salicylate, willow extract, beta hydroxybutanoic acid, tropic acid, and trethocanic acid, and (b) a hydroalcoholic gel dispersion, the hydroalcoholic gel dispersion including (1) at least one C2-C5 alcohol, (2) a blend of at least two powders selected from at least one inorganic powder and at least one nature-based organic powder, and (3) at least one nature-based polymer that excludes synthetic organic polymers; and a dispenser that includes a container that includes a retainer and an absorbent component disposed within the retainer, and a porous applicator that is in contact on a first side with the absorbent component in the retainer, and is exposed on a second side for contacting keratinous tissue to be treated, wherein the cosmetic composition is absorbed within the absorbent component and retained in the retainer, which retains the cosmetic composition in a dispersed gel state until dispensed; and wherein the dispenser optionally comprises a cap for covering the porous applicator when the dispensing system is not in use.

In some particular embodiments of the cosmetic dispensing system, the cosmetic composition includes a hydroalcoholic gel dispersion, comprising a blend of (i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers, (ii) at least one C2-C5 alcohol, (iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and (iv) a blend of at least two powders, at least one powder selected from inorganic powders and organic powders, and at least one nature-based organic powder, wherein the at least one beta hydroxy acid is dissolved in the hydroalcoholic gel dispersion and the blend of at least two powders is dispersed in the hydroalcoholic gel dispersion.

In some embodiments, the porous applicator of the cosmetic dispensing system includes one or more of: on at least the exposed surface intended for contacting skin, a plurality of pores having an average size in a range from about 30 to about 200 microns; on at least the first surface intended for contacting the sponge containing the cosmetic composition in the retainer, a plurality of pores having an average size in a range from about 90 to 200 microns; from the first surface to the second surface throughout the porous applicator, a plurality of pores having an average size in a range from 1 to about 1000 microns; the porosity through the porous material from the first surface to the second surface is uniform and continuous; the porosity through the porous material from the first surface to the second surface is variable from larger pores at the first surface to smaller pores at the second surface; and the porosity through the porous material from the first surface to the second surface is variable from smaller pores at the first surface to larger pores at the second surface.

In some embodiments, the invention also relates to enhancing the appearance of a keratinous material comprising using the applicator of the cosmetic system to apply the cosmetic composition of the cosmetic system to keratinous material.

In various embodiments, the disclosure provides a method for enhancing the appearance of acne prone skin, comprising: providing a dispensing system comprising: a cosmetic composition that has a gel crossover point suitable for dispensing through a porous applicator, and a dispenser that includes a container that includes a retainer and an absorbent component disposed within the retainer, and a porous applicator that is in contact on a first side with the absorbent component in the retainer, and is exposed on a second side for contacting keratinous tissue to be treated, wherein the cosmetic composition includes: (a) at least one beta hydroxy acid selected from -n-octanoylsalicylic acid, salicylate, sodium salicylate, willow extract, beta hydroxybutanoic acid, tropic acid, and trethocanic acid, and (b) a hydroalcoholic gel dispersion, the hydroalcoholic gel dispersion including: (1) at least one C2-C5 alcohol, (2) a blend of at least two powders selected from at least one inorganic powder and at least one nature-based organic powder, and (3) at least one nature-based polymer that excludes synthetic organic polymers; and wherein the dispenser containing the cosmetic composition is used to apply the composition to skin that is prone to acne by utilizing the dispenser to apply the cosmetic composition by contacting the porous applicator to skin using a dabbing motion to dispense a light coating of the cosmetic composition onto select areas of the skin in dispensed doses that are in an amount that is from about 0.01 mg to about 100 mg per application stroke.

In some particular embodiments of the method, the cosmetic composition includes a hydroalcoholic gel dispersion, comprising a blend of (i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers, (ii) at least one C2-C5 alcohol, (iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and (iv) a blend of at least two powders, at least one powder selected from inorganic powders and organic powders, and at least one nature-based organic powder, wherein the at least one beta hydroxy acid is dissolved in the hydroalcoholic gel dispersion and the blend of at least two powders is dispersed in the hydroalcoholic gel dispersion.

It is to be understood that both the foregoing general description and the following detailed description are exemplary and explanatory only and are not restrictive of the invention.

### BRIEF DESCRIPTION OF THE DRAWINGS

In accordance with the various embodiments, provided is

Features and advantages of the general inventive concepts will become apparent from the following description made with reference to the accompanying drawings, including drawings represented herein in the attached set of figures, of which the following is a brief description:

FIG. 1 shows a prior art example of a dispenser with a porous applicator;

FIG. 2 is a graphical representation of determining gel crossover point for an embodiment of the inventive cosmetic composition;

FIG. 3 is a graphical representation of determining gel crossover point for an embodiment of a comparative cosmetic composition;

FIG. 4 shows sol gel point properties of inventive compositions as a function of fractional percentage of nature-based polymers; and

FIG. 5 shows sweat resistance properties of inventive, comparative and control compositions.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

“Free of surfactant” means compositions that are free of, or substantially free of, or devoid of a surfactant. Some specific but non-limiting examples of emulsifiers that are lacking from the cosmetic composition includes surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di- C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific emulsifiers that are lacking from the cosmetic composition include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers.

“Gel Crossover Point” (Sol/Gel Point), means the point at which the G″ (loss modulus) intersects the G′ (storage modulus), which is reported in % strain. G* (complex modulus) provides insight on the flexibility and stiffness of a material. The cross-over point indicates the amount of shear that is required to collapse the microstructure (G″>G′) and allow the material to flow. It is the point at which a composition transitions from a more solid state to a more liquid state, such as from a gel state to a liquid state. An example of a method for determining gel crossover point is as follows: a Discovery HR-2 Rheometer by TA Instruments may be used, having 40 mm parallel plate geometry on a stainless-steel flat Peltier plate. The test can be run @ 20° C., with test parameter of angular frequency of 1.0 rad/s and logarithmic sweep: Strain % 0.01 to 1000.0%. 5 points per decade. Results are reported in % strain.

“Keratinous materials” includes materials containing keratin such as hair, skin, scalp, eyebrows, lips and nails.

“Nature-based” as used herein means and refers to cosmetically acceptable materials and components that are one or more of directly obtained from nature, are obtained from nature with minimal processing, and are derivatives of materials that are obtained from nature. Cosmetic compositions according to the instant disclosure are in some embodiments up to 99.11% natural.

“Soluble” and “Solubility” as used herein in reference to beta hydroxy acid means that the beta hydroxy acid is and remains substantially or fully dissolved in the hydroalcoholic gel.

“Stable” and “Stability” as used herein means that the cosmetic composition does not show any signs of significant changes in one or more of appearance, color, odor, pH, or viscosity at room temperature over at least a one-month period.

The cosmetic composition, dispensing systems and methods can comprise, consist of, or consist essentially of the essential elements and limitations of the invention described herein, as well as any additional or optional ingredients, components, or limitations described herein or otherwise useful.

The cosmetic composition is an on-the-go skin refresher that is especially good for acne-prone skin. The cosmetic composition can be used throughout the day to control shine and minimize skin imperfections and discourage acne and is suitable for use alone or over or under make up.

The cosmetic composition overcomes shortcomings in the art pertinent to salicylic acid induced instability in cosmetic compositions. Unexpectedly, according to the various embodiments herein and as exemplified in this disclosure, a combination of powder and polymer in a hydroalcoholic gel dispersion provides a composition that maintains salicylic acid solubility with up to about 2% salicylic acid while stably suspending oil absorbing powders. And in various embodiments the cosmetic composition exhibits a gel crossover point which allows it to pass through a porous applicator. With reference to the prior art, for example United States Patent Application Publication Number US20190231663A1 provides a cooling touch up gel formulation for over make up application that can be applied using a porous applicator and that includes a hydroalcoholic gel dispersion of powders. As shown in the comparative examples herein, exemplified compositions shown therein cannot be suitably modified by adding salicylic acid without compromising the sensorial and sol-gel properties of the composition. More particularly, the exemplified compositions therein demonstrate instability which is visible as separation into layers upon the addition of salicylic acid as disclosed in the examples herein below.

In some embodiments, the cosmetic composition is formulated for use in a dispenser that includes a container that includes a retainer and an absorbent component disposed within the retainer, and a porous applicator that is in contact on a first side with the absorbent component in the retainer, and is exposed on a second side for contacting keratinous tissue to be treated. Together the cosmetic composition and the dispenser comprise a cosmetic dispensing system. The dispenser may further comprise a cap or lid for covering the porous applicator when the dispensing system is not in use. The cosmetic composition is absorbed and retained in a retainer, which includes in some embodiments a sponge, and retains the cosmetic composition in a dispersed gel state until dispensed for application to skin. The dispenser is actuated to dispense the cosmetic composition onto skin by transfer first from the sponge to the porous applicator and then passage through the porous applicator toward the outer surface by capillary action. In some embodiments, the actuation may be achieved by compression of the exposed side of the porous applicator. According to the process of dispensing, the cosmetic composition must convert from a gel to a liquid in order to pass through the pores of the applicator by capillary action. The specific property of the gel form of the cosmetic composition is characterized by its “gel crossover point” which must be within a range that is suitable to allow passage through the pores of the porous applicator while maintain suspension of the powders and stable solubility of the salicylic acid.

In accordance with the exemplified embodiments, the cosmetic composition demonstrates effective reduction and prevention of oil and shine, control and reduction of acne and breakouts, which is delivered with a refreshing, cool skin feel, and is gentle on skin and does not disturb makeup.

Cosmetic Composition

According to the present invention, the cosmetic composition comprises, in various embodiments: (a) at least one beta hydroxy acid in a (b) gel dispersion, the gel dispersion comprising at least one each of (i) a C2-C5 alcohol, (ii) a blend of inorganic and organic/nature-based powders, (iii) a nature-based polymer. In some embodiments, the cosmetic composition may be dispensed using a porous applicator and is applied as an on-the-go skin refresher that is especially good for acne-prone skin. The cosmetic composition can be used throughout the day to control shine and minimize skin imperfections and discourage acne and is suitable for use alone or over or under make up.

In some particular embodiments, the cosmetic composition includes a hydroalcoholic gel dispersion, comprising a blend of (i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers, (ii) at least one C2-C5 alcohol, (iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and (iv) a blend of at least two powders, at least one powder selected from inorganic powders and organic powders, and at least one nature-based organic powder, wherein the at least one beta hydroxy acid is dissolved in the hydroalcoholic gel dispersion and the blend of at least two powders is dispersed in the hydroalcoholic gel dispersion.

In some embodiments, the cosmetic composition is free of, or substantially free of, or devoid of one or more of surfactants, oils, silicones, and film formers.

In some embodiments, the cosmetic composition has a gel crossover point ranging from about 5% to about 85% strain. In some embodiments from about 20% to about 80% strain, and in some embodiments from about 35% to about 75% strain, including all ranges and subranges there between.

In accordance with the various embodiments, the cosmetic composition is suitable for application to keratinous tissue any one or more of directly, under make up, and over make up. For purposes hereof, a “make up” as referred to herein can be any cosmetic composition utilized for application to a keratinous tissue for conferring at least an aesthetic effect, and in some examples containing a coloring agent which can provide color to a keratinous material to which it has been applied.

Beta Hydroxy Acid Component

In accordance with the various embodiments, the cosmetic composition comprises in the gel dispersion a component that comprises at least one beta hydroxy acid.

In some particular embodiments according to the disclosure, the composition includes salicylic acid present in the cosmetic composition in an amount that ranges from about 0.2% to about 1% by weight, based on the total weight of the composition, or from about 0.2% to about 2% by weight, based on the total weight of the composition.

The total amount of beta hydroxy acid includes in some embodiments from about 0.2% up to and not more than about 2% of beta hydroxy acid, by weight, based on the total weight of the cosmetic composition. In some embodiments, the cosmetic composition includes and up to and not more than about 1.9% of beta hydroxy acid. In some embodiments, the cosmetic composition includes from about 0.2% to about 1% of beta hydroxy acid. In some embodiments, the cosmetic composition includes and up to and not more than about 1% of beta hydroxy acid.

The term “beta-hydroxy acid” is understood to mean, according to the present invention, a carboxylic acid having a hydroxyl functional group and a carboxylic functional group separated by two carbon atoms. A beta hydroxy acid can be present in the cosmetic composition in the form of the free acid and/or in the form of one of its associated salts (salts with an organic base or an alkali metal, in particular), especially according to the final pH imposed on the composition.

The beta hydroxy acid is selected from one or more of salicylic acid and derivatives thereof (including 5-n-octanoylsalicylic acid, salicylate, sodium salicylate, and willow extract), beta hydroxybutanoic acid, tropic acid, and trethocanic acid.

In some embodiments, the composition includes a beta hydroxy acid comprising salicylic acid.

In accordance with the various embodiments, the amount of beta-hydroxy acid present in the compositions is in the range from about 0.2% to about 2%, and in some embodiments from about 0.2% to about 1.5%, and in some embodiments from about 0.2% to about 1%, including increments and all ranges and subranges therein and there between, by weight, based on the total weight of the composition. One of ordinary skill in the art, however, will appreciate that other ranges are within the scope of the invention.

Thus, the at least one beta hydroxy acid is present in the cosmetic composition from about 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

Alcohol Component

In accordance with the various embodiments, the cosmetic composition comprises in the gel dispersion a component that comprises at least one alcohol. In addition, in some embodiments, the cosmetic composition may comprise at least one cooling component.

C2-C5 Alcohol and Water (Hydro-Alcoholic Solvent Component)

In some embodiments, the cosmetic composition comprises at least one C2-C5 alcohol. Suitable C2-C5 alcohols include ethanol, propanol, butanol, pentanol, isopropanol, isobutanol and isopentanol. In some embodiments the at least one alcohol is denatured. In some particular embodiments, the at least one alcohol comprises ethanol.

In some particular embodiments according to the disclosure, the composition includes alcohol, for example, denatured alcohol, present in the cosmetic composition in an amount that ranges from about 5% to about 20% by weight, based on the total weight of the composition. In some particular embodiments, denatured alcohol is present in the cosmetic composition in an amount that is about 20% by weight.

In some embodiments, the at least one alcohol is/are present in the cosmetic composition in amounts ranging from about 5% to about 30%, and in some embodiments from about 5% to about 25%, and in some embodiments from about 5% to about 20%, and in some embodiments from about 10% to about 15%, including increments and all ranges and subranges therein and there between, by weight, based on the total weight of the composition. One of ordinary skill in the art, however, will appreciate that other ranges are within the scope of the invention. Thus, the least one alcohol is present in the cosmetic composition from about 5, 6, 7, 8, 9, 10, 11, 12, 13, 14, 15, 1, 17, 18, 19, 20, 25, to about 30 weight percent, including increments and all ranges and subranges therein and there between.

In accordance with the various embodiments, the cosmetic composition includes water.

In various embodiments, the cosmetic composition comprises from about 5% to about 90% water, and in some embodiments from about 20% to about 85% water, and in some embodiments from about 50% to about 85% water, and in some embodiments from about 60% to about 80% water, and in some embodiments from about 65% to about 75%, water, including increments and all ranges and subranges therein and there between, by weight, based on the total weight of the composition. One of ordinary skill in the art, however, will appreciate that other ranges are within the scope of the invention. Thus, water is present, by weight, based on the total weight of the composition, from about 5, 10, 15, 20, 25, 30, 35, 40, 45, 50, 55, 60, 65, 66, 67, 68, 69, 70, 71, 72, 73, 74, 75, 80, 85, to about 90 weight percent, including increments and all ranges and subranges therein and there between.

The water used in the cosmetic composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cosmetic composition is in the range from about 1.0 to about 5.0, and in some embodiments, is in the range from about 3.5 to about 4.5, and in some embodiments in the range from about 3.9 and 4.5, and in some embodiments in the range from about 4.1 to about 4.4, and in some embodiments the pH is about 4.2. Generally, the pH can be adjusted to the desired value by addition of a base or an acid (organic or inorganic). In view of the inclusion of hydroxy acids in the cosmetic composition hereof, the pH is typically adjusted using a base, which in some examples is sodium hydroxide, or using an acid, which in some examples is citric acid.

Powder Component

In accordance with the various embodiments, the cosmetic composition comprises in the gel dispersion a component that comprises at least one powder. In some embodiments, the cosmetic composition comprises at least two powders, and in some embodiments the cosmetic composition comprises three or more powders.

In some embodiments, the cosmetic composition comprises at least one inorganic powder and at least one organic powder. In some particular embodiments, the composition includes at least one inorganic powder and two, three or more nature-based organic powders. In some such embodiments, the cosmetic composition comprises powders that include perlite, and microcrystalline cellulose. In some particular embodiments, the cosmetic composition comprises at least one inorganic and one organic powder selected from perlite, microcrystalline cellulose, cellulose, and kaolin.

In various embodiments, the composition excludes non-natural powders that are selected from polyethylene beads or powder, such as those sold under the name Microthene MN 727 or Microthene MN 710-20 by Equistar or such as the powders sold under the name Gotalene 120 Colorless 2 by Dupont; thermoplastic powders such as Nylon particles, such as the Nylon-12 sold by Arkema under the name Orgasol 2002 Exd Nat Cos, spherical organic powders, for example, thermoplastic powders such as Nylon particles, such as the Nylon-12 sold by Arkema under the name Orgasol 2002 Exd Nat Cos; polyethylene powders; polytetrafluoroethylene (Teflon®) powders; microspheres based on acrylic copolymers, such as those made of ethylene glycol dimethacrylate/lauryl methacrylate copolymer sold by Dow Corning under the Polytrap name; expanded powders, such as hollow microspheres and in particular the microspheres sold under the Expancel name by Kemanord Plast or under the name Micropearl F 80 ED by Matsumoto; silicone resin microbeads, such as those sold under the Tospearl name by Toshiba Silicone; polymethyl methacrylate microspheres, sold under the name Microsphere M-100 by Matsumoto or under the name Covabead LH85 by Wackherr; ethylene/acrylate copolymer powders, such as those sold under the Flobeads name by Sumitomo Seika Chemicals, silicone resin microbeads (Tospearl® from Toshiba, for example); polyurethane powders, in particular powders formed of crosslinked polyurethane comprising a copolymer, the said copolymer comprising trimethylol hexyllactone, such as the hexamethylene diisocyanate/trimethylol hexyllactone polymer sold under the name Plastic Powder D-400® or Plastic Powder D-800® by Toshiki; carnauba microwaxes, such as that sold under the name MicroCare 350® by Micro Powders; synthetic wax microwaxes, such as that sold under the name MicroEase 114S® by Micro Powders; microwaxes composed of a mixture of carnauba wax and polyethylene wax, such as those sold under the names Micro Care 300® and 310® by Micro Powders; microwaxes consisting of a mixture of carnauba wax and synthetic wax, such as that sold under the name Micro Care 325® by Micro Powders; or polyethylene microwaxes, such as those sold under the names Micropoly 200®, 220®, 220L® and 250S® by Micro Powders, and silica silylate aerogels.

In some particular embodiments, the at least one powder excludes one or more of microplastics, silica and aerogels (silica silylate).

In some embodiments, at least two powders are selected wherein the powders vary in particulate size.

In some particular embodiments according to the disclosure, the composition includes at least one inorganic powder and at least one organic and/or organic nature-based powder. According to such embodiments, each of the at least one inorganic powder is present in the cosmetic composition in an amount that ranges from about 0.5% to about 1% by weight, based on the total weight of the composition. In some particular embodiments, the inorganic powder is present in the cosmetic composition in an amount that is about 0.8% by weight. Further according to such embodiments, each one of the at least one organic powder is present in the cosmetic composition in an amount that ranges from about 1.4% to about 2% by weight, based on the total weight of the composition. In some such particular embodiments, the at least one organic powder is present in the cosmetic composition in an amount that is about 1.6% by weight and may comprise microcrystalline cellulose. Further according to such embodiments, each one of the at least one organic nature-based powder is present in the cosmetic composition in an amount that ranges from about 0.2% to about 1.25% by weight, based on the total weight of the composition. In some such particular embodiments, the at least one organic nature-based powder is present in the cosmetic composition in an amount that is about 0.25% or about 1% by weight and may comprise one or more of cellulose and kaolin. According to some such embodiments, the powders include perlite, microcrystalline cellulose, cellulose, and kaolin.

“Powder” should be understood as meaning solid particles which are insoluble in the medium of the cosmetic composition, whatever the temperature at which the cosmetic composition is manufactured. The powder component includes an oil absorbing powder.

In the various embodiments, the at least one powder is selected from particles of mineral, vegetable or organic origin.

The powders can be colorless or white and inorganic or organic, of any physical shape (platelet, spherical or oblong) and of any crystallographic form (for example sheet, cubic, hexagonal, orthorhombic and the like). The powders can be porous or nonporous.

In some embodiments, powders are selected from inorganic materials such as perlite; poly(vinyl chloride) powder; pumice (INCI name) such as pumice 3/B from Eyraud; ground fruit kernel shells, such as ground materials derived from apricot kernels or walnut shells; sawdust, wood flour or cork flour; glass beads; alumina (aluminium oxide) (INCI name: Alumina), such as the product sold under the name Dermagrain 900 by Marketech International; sugar crystal.

In some embodiments, the at least one powder is selected from particles comprising microcrystalline cellulose and cellulose.

In some embodiments, the at least one powder is selected from particles based on mannitol and cellulose which are sold under the Unisphere names by Induchem, agar-based capsules which are sold under the Primasponge names by Cognis and spheres based on jojoba esters which are sold under the Floraspheres names by Floratech; and their mixtures. I

In some embodiments, the at least one powder is selected from particles formed of natural organic materials, such as starch powders, in particular powders formed of maize, wheat or rice starch, which are or are not crosslinked, such as powders formed of starch crosslinked with octenylsuccinic anhydride, sold under the Dry-Flo name by National Starch.

In some embodiments, each one of the at least one powder is present in the cosmetic composition in amounts ranging from about 0.005% to about 10%, in some embodiments from about 0.05% to about 7.5%, in some embodiments from about 0.5% to about 5%, by weight, and in some embodiments from about 0.75% to about 2%, including increments and all ranges and subranges therein and there between, by weight, based on the total weight of the composition. One of ordinary skill in the art, however, will appreciate that other ranges are within the scope of the invention. Thus, each one of the at least one powder is present in the cosmetic composition from about 0.005, 0.01, 0.05, 0.1, 0.2, 0.3, 0.4, 0.5, 1, 1.5, 2, 2.5, 3, 3.5, 4, 4.5, 5, 6, 7, 8, 9, to about 10 weight percent, including increments and all ranges and subranges therein and there between.

In some embodiments, the total combined amount of powder(s) present in the cosmetic composition is less than 12% by weight with respect to the total weight of the cosmetic composition, including in the range from about 0.5% to about 10%, in some embodiments from about 1% to 8%, in some embodiments from about 1% to about 3%, and in some embodiments from about 1.5% to about 2.5%, including increments and all ranges and subranges therein and there between, by weight, based on the total weight of the composition. One of ordinary skill in the art, however, will appreciate that other ranges are within the scope of the invention. Thus, the total combined amount of powder(s) including silica aerogel(s) present in the cosmetic composition is present from about 0.5, 0.6, 0.7, 0.8, 0.9, 1.0, 2, 3, 4, 5, 6, 7, 8, 9, 10, 11, to about 12 weight percent, including increments and all ranges and subranges therein and there between.

Polymer Component

In accordance with the various embodiments, the cosmetic composition comprises in the gel dispersion a component that comprises at least one nature-based polymer. In the various embodiments the at least one nature-based polymer is not electrolyte sensitive or salt intolerant, such that the at least one polymer is suitable to maintain suspension/dispersion of the at least one powder at low pH and in the presence of salts/electrolytes. Thus, the cosmetic composition is free of polymers that are electrolyte sensitive/salt intolerant, which polymers are susceptible to incomplete swelling in water or other solvents and may collapse/break in the presence of electrolytes and will result in a low/no viscosity gel that is incapable of suspending the powders.

In some particular embodiments according to the disclosure, the composition includes at least one nature-based polymer present in the cosmetic composition in an amount that ranges from about 0.2% to about 0.6% by weight, based on the total weight of the composition. In some particular embodiments, each one of the nature-based polymer is present in the cosmetic composition in an amount that ranges from about 0.25% to about 0.5%, and the total amount of nature-based polymer is present in the cosmetic composition in an amount that is in the range from about 0.5% to about 1% by weight, and in some embodiments is about 0.75% by weight. In some particular embodiments, the composition includes two or more and in some embodiments three nature-based polymers. According to some such embodiments, the cosmetic composition includes each of a blend of xanthan gum and sclerotium gum present in the cosmetic composition in an amount of about 0.5% by weight, and hydroxypropyl guar present in the cosmetic composition in an amount of about 0.25% by weight.

In accordance with the various embodiments, the cosmetic composition comprises in the gel dispersion a component that comprises at least one nature-based polymer and excludes synthetic organic polymers, for example synthetic/man-made thermoplastics, thermosets, elastomers, and synthetic fibers.

In some particular embodiments, the cosmetic composition excludes one or all of non-nature-based polymers, for example, but not limited to polymers selected from amphiphilic polymers that include, but are not limited to, hydrophobically-modified sulfonic acid copolymers such as Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Aristoflex SNC (crosslinked ethoxylated AMPS/C16-C18), and Aristoflex LNC (noncrosslinked AMPS/C12-C14).

In some particular embodiments, the cosmetic composition excludes one or more of polymers selected from dilinoleic acid/butanediol copolymer, dimer dilinoleyl dimer dilinoleate, algin, oxidized starch acetate, cyclodextrin, hydroxypropyl guar hydroxypropyltrimonium chloride, sodium dextran sulfate, polyhydroxylstearic acid, microcrystalline cellulose (and) cellulose gum, biosaccharide gum-1 (and) sodium levulinate (and) glyceryl caprylate (and) sodium anisate, and mannitol (and) cellulose (and) hydroxypropyl methyl cellulose.

In some particular embodiments, the compositions expressly exclude synthetic organic polymers selected from Ammonium Acryloyldimethyltaurate/Steareth-25 Methacrylate Crosspolymer, polyacrylate crosspolymer 6 and acrylates/C10-30 Alkyl acrylate crosspolymer.

Regarding nature-based polymers, mention may be made broadly of polysaccharides, including but not limited to alginate, hyaluronan, dextran, starch, cellulose derivatives, chitin derivatives; proteins, including collagen, gelatin, fibrin, elastin, silk fibroin, soy protein; bacterial polyesters, for example polymers and copolymers of hydroxyalkanoic acids.

In some embodiments according to the disclosure, the at least nature based polymer is selected from natural gums and alginates, for example acacia Senegal gum, Caesalpinia spinosa gum, agar, carob gum, carrageenan, carrageenan (lambda, iota kappa), cellulose gum, corn starch modified, dehydroxanthan gum, gellan gum, guar gum, guar hydroxypropyltrimonium chloride, gum acacia, gum arabic, gum ghatti, gum karaya, gum tragacanth, hydroxypropyl guar, hydroxypropyl methyl cellulose, lecithin, microcrystalline cellulose, pectin, potato starch modified, pullulan, sclerotium gum, sodium carboxymethyl starch, sorbitol/sebacic acid copolymer, xanthan gum, and mixtures thereof. In some embodiments, the at least one nature based polymer is selected from one or a combination of pullulan, xanthan gum, hydroxypropyl guar, sclerotium gum. In some particular embodiments, the at least one nature based polymer includes the combination of pullulan, xanthan gum, hydroxypropyl guar, lecithin, and sclerotium gum. In some particular embodiments, the at least one nature-based polymer includes the combination of xanthan gum, hydroxypropyl guar, and sclerotium gum.

In some embodiments of the cosmetic composition suitable for delivery in a dispenser system according to the disclosure, the nature-based polymer of the composition includes a blend of at least two nature-based polymers selected from a blend of xanthan gum and sclerotium gum, and hydroxypropyl guar, and further includes alcohol present in an amount that is at least 20% by weight, based on the weight of the composition, and further includes a blend of at least one inorganic and one organic powder selected from perlite, microcrystalline cellulose, cellulose, and kaolin.

In some embodiments of the cosmetic composition suitable for delivery in a dispenser system according to the disclosure, the nature-based polymer of the composition may include a combination of polymers comprising xanthan gum, hydroxypropyl guar, and sclerotium gum.

In some embodiments of the cosmetic composition suitable for delivery in a dispenser system according to the disclosure, the nature-based polymer of the composition excludes the polymers that include dilinoleic acid/butanediol copolymer, dimer dilinoleyl dimer dilinoleate, algin, oxidized starch acetate, cyclodextrin, hydroxypropyl guar hydroxypropyltrimonium chloride, sodium dextran sulfate, polyhydroxylstearic acid, microcrystalline cellulose (and) cellulose gum, biosaccharide gum-1 (and) sodium levulinate (and) glyceryl caprylate (and) sodium anisate, and mannitol (and) cellulose (and) hydroxypropyl methyl cellulose.

In accordance with the disclosure, selection of the at least nature based polymer may be achieved through a series of tests (“Progression Experiments”) which demonstrate that: the polymer has suspending capability; the polymer is tolerant to salt, including beta hydroxy acid (salicylic acid) and sodium hydroxide; the polymer can be dispersed in water and alcohol to form a homogeneous hydroalcoholic dispersion that is free or substantially free of non-dispersed gel bodies; and the polymer must suspend the at least one powder without separation. Embodiments of the cosmetic composition that satisfy all of the test criteria are candidates for selection in porous applicators and are further tested for gel-sol point, viscosity, ability to re-gel. The Examples provided herein demonstrate formulations that fail one or more of the tests and also exemplify inventive compositions that are suitable for maintaining solubility of high salicylic acid in a stable gel dispersion of powders, an suitability for use in a dispensing system comprising a porous applicator with a sponge retainer.

In some embodiments, the at least one nature-based polymer is present in the cosmetic composition in amounts ranging from about 0.05% to about 5%, in some embodiments from about 0.1% to about 1%, and in some embodiments from about 0.2% to about 0.5%, including increments and all ranges and subranges therein and there between, by weight, based on the total weight of the composition. One of ordinary skill in the art, however, will appreciate that other ranges are within the scope of the invention. Thus, the at least one nature-based polymer is present in the cosmetic composition from about 0.05. 0.1, 0.15, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.5, 3.0, 3.5, 4.0, 4.5 to about 5.0 weight percent, including increments and all ranges and subranges therein and there between.

Optional Additives

The cosmetic composition may also include one or more additives used in the cosmetics field which do not affect the properties of the cosmetic composition according to the invention, such as one or more of any of an antioxidant, vitamin, essential oil, botanical extract, pH adjuster/neutralizing agent, fragrance, odor absorber, preservative, an anti-microbial, including, but not limited to, phenoxyethanol, chlorphenesin, capryloyl glycol, and combinations thereof. In some embodiments, the cosmetic composition includes additives selected from fragrance, and phenoxyethanol. In some embodiments, the cosmetic composition includes additives selected from fragrance.

Although the optional additives are given as examples, it will be appreciated that other optional components compatible with cosmetic applications known in the art may be used.

In accordance with the various embodiments, the amount of each additive, when present in the cosmetic composition, can be present in a range from about 0.001% to about 20%, and in some embodiments from about 0.005% to about 0.01%, and in some embodiments from about 0.01% to about 0.1%, and in some embodiments from about 0.15% to about 5%, and in some embodiments from about 0.40% to about 4%, and in some embodiments from about 0.5% to about 2.5%, and in some embodiments from about 1% to about 2%, including increments and all ranges and subranges therein and there between, by weight, based on the total weight of the composition. One of ordinary skill in the art, however, will appreciate that other ranges are within the scope of the invention. And in some embodiments, a combination of additives present in the cosmetic composition can be present in a range from about 0.001% to about 20%. Thus, any one or a combination of additives may be present in the cosmetic composition from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and all ranges and subranges therein and there between.

Cosmetic Dispensing System

In some embodiments of the cosmetic composition according to the disclosure, the cosmetic composition may, as desired, be selected for use with a cosmetic dispensing system that includes a porous applicator. Of course, it will be appreciated that while some embodiments of the cosmetic composition may be suited for use with a porous applicator as described herein, such use is not a required aspect of the instant invention. Thus, it will be further appreciated that embodiments of the cosmetic composition may not be suited for use with the applicator. Thus, according to the present invention, the cosmetic composition hereof may be selected for use as a component of a cosmetic dispensing system which comprises, in various embodiments, a cosmetic composition that has a gel crossover point suitable for dispensing through a porous applicator, and a dispenser that includes a container that includes a retainer and an absorbent component disposed within the retainer, and a porous applicator comprising a porous material that is in contact on a first side with the absorbent component in the retainer, and is exposed on a second side for contacting keratinous tissue to be treated. The dispenser may further comprise a cap or lid for covering the porous applicator when the dispensing system is not in use. The dispenser may have any shape or size, and thus may be provided in a container design that is similar to any of a variety of cosmetic applicators having shapes that are cylindrical, hemispherical or spherical, block shaped, tubular, pencil shaped, and the like. It will be appreciated that the shape is not limiting. And the material forming each of the container, including the retainer, the absorbent component, and the porous applicator may be formed of any one or combination of plastic, natural fibers, synthetic fibers, ceramic, metal, natural sponge or other nature-based material and the like.

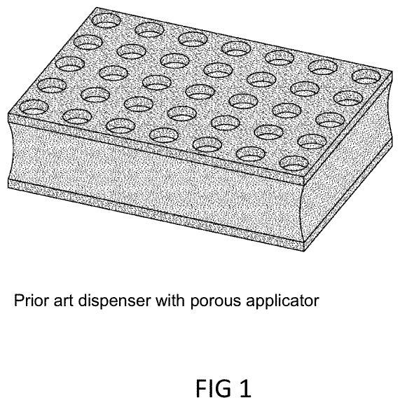

Reference may be made to PCT application WO2018152472A1 which discloses a liquid applicator for applying a liquid or gel to a surface. The liquid applicator comprises a sintered porous elastomeric material body. Liquid moves from one end of the applicator through to another end of the liquid applicator by pushing the liquid through the applicator. The sintered porous elastomeric body can be selected from the group consisting of ethylene vinyl acetate, polypropylene, polyethylene for example high density polyethylene, low density polyethylene or ultrahigh molecular weight polyethylene and has an average pore size greater than 20 microns, greater than 40 microns, greater than 60 microns, greater than 80 microns, greater than 100 microns, greater than 125 microns, greater than 150 microns, greater than 175 microns, greater than 200 microns, or greater than 250 microns. In some embodiments, the sintered porous elastomeric body has an average pore size less than about 300 microns. Referring to the instant drawings, FIG. 1 shows a representative embodiment of a dispenser that is disclosed in the above identified PCT application as FIG. 23.

The cosmetic composition is absorbed and retained in the retainer, which includes in some embodiments a sponge, and retains the cosmetic composition in a dispersed gel state until dispensed for application to skin. The dispenser is actuated to dispense the cosmetic composition onto skin by transfer first from the sponge to the porous applicator and then passage through the porous applicator toward the outer surface by capillary action. In some embodiments, the actuation may be achieved by compression of the exposed side of the porous applicator. According to the process of dispensing, the cosmetic composition must convert from a gel to a liquid in order to pass through the pores of the applicator by capillary action. The specific property of the gel form of the cosmetic composition is characterized by its gel crossover point which must be within a range that is suitable to allow passage through the pores of the porous applicator while maintain suspension of the powders and stable solubility of the salicylic acid.

In embodiments suitable for use with the cosmetic dispensing system, the cosmetic composition has a gel crossover point ranging from about 5% to about 85% strain. In some embodiments from about 20% to about 85% strain, in some embodiments from about 25% to about 80%, and in some embodiments from about 35% to about 75% strain, including all ranges and subranges there between. In the various embodiments of the cosmetic composition that is formulated for delivery using a porous applicator has a gel crossover point which allows it to suspend the at least one powder and pass through a porous applicator with shear. Thus, in some embodiments, of the cosmetic composition that is formulated for delivery using a porous applicator, the cosmetic composition is formulated to have a gel crossover point that is suited to the pore size of an applicator.

In some particular embodiments the cosmetic composition excludes one or all of non-nature-based polymers, for example, but not limited to polymers selected from Ammonium Acryloyldimethyltaurate/Steareth-25 Methacrylate Crosspolymer, polyacrylate crosspolymer 6 and acrylates/C10-30 Alkyl acrylate crosspolymer.

In some embodiments of the cosmetic composition suitable for delivery in a dispenser system according to the disclosure, the nature-based polymer of the composition includes a blend of at least two nature-based polymers selected from a blend of xanthan gum and sclerotium gum, and hydroxypropyl guar, and further includes alcohol present in an amount that is at least 20% by weight, based on the weight of the composition, and further includes a blend of at least one inorganic and one organic powder selected from perlite, microcrystalline cellulose, cellulose, and kaolin.

In some embodiments of the cosmetic composition suitable for delivery in a dispenser system according to the disclosure, the nature-based polymer of the composition may include a combination of polymers comprising xanthan gum, hydroxypropyl guar, and sclerotium gum.

In some embodiments of the cosmetic composition suitable for delivery in a dispenser system according to the disclosure, the nature-based polymer of the composition excludes the polymers that include dilinoleic acid/butanediol copolymer, dimer dilinoleyl dimer dilinoleate, algin, oxidized starch acetate, cyclodextrin, hydroxypropyl guar hydroxypropyltrimonium chloride, sodium dextran sulfate, polyhydroxylstearic acid, microcrystalline cellulose (and) cellulose gum, biosaccharide gum-1 (and) sodium levulinate (and) glyceryl caprylate (and) sodium anisate, and mannitol (and) cellulose (and) hydroxypropyl methyl cellulose.

In embodiments suitable for use with the cosmetic dispensing system, the porous applicator of the cosmetic dispensing system includes on at least the exposed surface intended for contacting skin, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns. In some embodiments, the porous applicator of the cosmetic dispensing system includes, on at least the first surface intended for contacting the sponge containing the cosmetic composition in the retainer, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns, and the portion of the porous material at the bottom of the base portion of the container has an average pore size of about 90 to 200 microns. In some embodiments, the porous applicator of the cosmetic dispensing system includes from the first surface to the second surface throughout the porous material, a plurality of pores having an average size in a range from 1 to about 1000 microns, and in some embodiments, in a range from about 10 to about 50 microns, and in some embodiments in a range from about 25 microns to about 250 microns, and in some embodiments in a range from about 30 to about 100 microns., and the portion of the porous material at the bottom of the base portion of the container has an average pore size of about 90 to 200 microns.

Thus, in various embodiments, pores on any one surface or through the entire material from the first surface to the second surface have a pore size in the range from about 1, 2, 3, 4, 5, 6, 7, 8, 9, 10, 20, 30, 40, 50, 60, 70 80, 90, 100, 200, 300, 400, 500, 600, 700, 800, 900 to about 1000 microns, including increments and all ranges and subranges therein and there between According to some embodiments, the pores are interconnecting. According to the various embodiments, the porosity through the material from the first surface to the second surface is one of uniform and continuous, variable from larger pores at the first surface to smaller pores at the second surface, and variable from smaller pores at the first surface to larger pores at the second surface.

The porous material may be formed of any suitable material that may be one or a combination of gel-foam, polymeric, ceramic, metal and the like.

Methods for Enhancing the Appearance of Acne Prone Skin

According to the present invention, the method for enhancing the appearance of acne prone skin comprises, in various embodiments, includes providing a dispensing system as disclosed herein wherein the dispenser containing the cosmetic composition is used to apply the composition to skin that is prone to acne. The dispenser system may be utilized to apply the cosmetic composition by stroking using a dabbing motion to dispense a light coating of the cosmetic composition onto select areas of the skin in dispensed doses that are in an amount that is from about 0.01 mg to about 100 mg of the cosmetic composition, and in discrete doses per dab in a range from about 0.01 mg to about 100 mg/dab, or from about 0.25 mg to about 25 mg per dab, or from about 0.5 mg to about 20 mg/dab, or from about 1 mg to about 15 mg/dab, or from about 2 mg to about 10 mg/dab. In some embodiments, a single use of the cosmetic composition will include application of about 1-10 dabs or about 2-6 dabs, and in some embodiments about 4 dabs. Thus, in some embodiments, a dose application of the cosmetic composition includes about 4 dabs at a concentration per dab in the range from about 0.25 mg-25 mg. In one example a dose application may be 1 mg up to about 100 mg, which may be delivered in a single application stroke or in more than one application stroke.

### EXAMPLES

### Example 1: Raw Materials

The raw materials as used in the inventive composition include salicylic acid, perlite, microcrystalline cellulose (Commercial reference Cellulobeads USF from Daito Kasei Kogyo, Commercial reference is Tego Feel C 10 from Evonik Goldschmidt), alcohol denat., sclerotium gum (and) xanthan gum, and hydroxypropyl guar.

### Example 2: Inventive Composition

Exemplary embodiments of the inventive composition is set forth in Table 1, below.

TABLE 1
Inventive compositions

| INGREDIENT | INV 1 | INV 2 | INV 3 | INV 4 | INV 5 | INV 6 | INV 7 |
|---|---|---|---|---|---|---|---|
| WATER | 76.25 | 75.25 | 76 | 75.25 | 76 | 75.25 | 76 |
| SCLEROTIUM GUM (and) | 0.5 | 0.5 | 0.5 | 0.5 | 0.5 | 0.5 | 0.5 |
| XANTHAN GUM | | | | | | | |
| HYDROXYPROPYL GUAR | 0.25 | 0.25 | 0.25 | 0.25 | 0.25 | 0.25 | 0.25 |
| ALCOHOL DENAT. | 20 | 20 | 20 | 20 | 20 | 20 | 20 |
| SALICYLIC ACID | 0.5 | 0.5 | 0.5 | 0.5 | 0.5 | 0.5 | 0.5 |
| SODIUM HYDROXIDE | 0.1 | 0.1 | 0.1 | 0.1 | 0.1 | 0.1 | 0.1 |
| PERLITE | 0.8 | 0.8 | 0.8 | 0.8 | 0.8 | 0.8 | 0.8 |
| MICROCRYSTALLINE | 1.6 | 1.6 | 1.6 | 1.6 | 1.6 | 1.6 | 1.6 |
| CELLULOSE | | | | | | | |
| SODIUM HYDROXIDE | 0 | 0 | 0 | 0 | 0 | 0 | 0 |
| CELLULOSE** | | 1 | 0.25 | | | | |
| CELLULOSE | | | | 1 | 0.25 | | |
| KAOLIN | | | | | | 1 | 0.25 |
| Viscosity (M2, 24 h), uD | 24.7 | 26.7 | | | | | |
| Crossover modulus, % | 69.2 | | | | | | |

### Example 3: Comparative Compositions

Comparative composition are set forth in Table 2, below.

TABLE 2
Comparative Compositions

| INGREDIENT | COMP 1 | COMP 3 | COMP 4 |
|---|---|---|---|
| WATER | 69.96 | 76 | 77.75 |
| AMMONIUM | 0.35 | | 1.13 |
| ACRYLOYLDIMETHYLTAURATE/ | | | |
| STEARETH-25 METHACRYLATE | | | |
| CROSSPOLYMER | | | |
| ALCOHOL DENAT. | 25 | 20 | 20 |
| METHYL DIISOPROPYL | 0.2 | | 0.2 |
| PROPIONAMIDE | | | |
| | 0.1 | | |
| TRIETHANOLAMINE | 0 | | |
| PERLITE | 0.63 | 0.8 | |
| CELLULOSE | 2 | | |
| MICROCRYSTALLINE CELLULOSE | 1.5 | 1.6 | |
| SILICA SILYLATE | 0.26 | | |
| SALICYLIC ACID | | 0.5 | 0.5 |
| SODIUM HYDROXIDE | | 0.1 | 0.12 |
| CITRIC ACID | | | |
| FRAGRANCE/PARFUM | | | 0.3 |
| SCLEROTIUM GUM (and) | | 0.5 | |
| XANTHAN GUM | | | |
| XANTHAN GUM (and) | | 0.25 | |
| SCLEROTIUM GUM (and) LECITHIN | | | |
| (and) PULLULAN | | | |
| HYDROXYPROPYL GUAR | | 0.25 | |
| | No anti-acne | Comparative | Inferior sweat |
| | active FROM US | natural formula | resistance |
| | 20190231663 | but clogs porous | |
| | | applicator | |

### Example 4. Nature-Based Polymer Testing

Natural Polymer Testing

TABLE 3
Formulation for testing natural polymers

| RAW MATERIAL INCI | W/W % FORMULA |
|---|---|
| salicylic acid | 0.5 |
| sodium hydroxide | 0.12 |
| perlite | 0.8 |
| microcrystalline cellulose | 1.6 |
| alcohol denaturant | 20 |
| natural polymer(s) (see below) | see below |
| water | QS |

TABLE 4
Key to Polymers Combinations Tested in formulation shown in Table 3

| POLYMER | Polymer Name |
|---|---|
| FORMULATION # | (w/w %; raw materials are separated by semicolons) |
| POLY 1 | xanthan gum (0.5); hydroxypropyl guar (0.5) |
| POLY 2 | xanthan gum (0.25); hydroxypropyl guar (0.25) |
| POLY 3 | xanthan gum (0.1); hydroxypropyl guar (0.1) |
| POLY 4 | xanthan gum (0.5) |
| POLY 5 | xanthan gum (0.2) |
| POLY 6 | xanthan gum (0.1) |
| POLY 7 | hydroxypropyl guar (0.5) |
| POLY 8 | xanthan gum (0.22), sclerotium gum (0.1), lecithin (0.095), and pullulan |
| | (0.095) |
| POLY 9 | xanthan gum (0.13), sclerotium gum (0.06), lecithin (0.057), and pullulan |
| | (0.057); hydroxypropyl guar (0.7) |
| POLY 10 | xanthan gum (0.22), sclerotium gum (0.1), lecithin (0.095), and pullulan |
| | (0.095); hydroxypropyl guar (0.5) |
| POLY 11 | xanthan gum (0.33), sclerotium gum (0.15), lecithin (0.143), and pullulan |
| | (0.143); hydroxypropyl guar (0.25) |
| POLY 12 | gellan gum (0.5) |
| POLY 13 | gellan gum (0.25) |
| POLY 14 | corn starch (0.5) |
| POLY 15 | cellulose gum (0.5) |
| POLY 16 | pullulan (1) |
| POLY 17 | hydroxypropyl starch phosphate (0.5) |
| POLY 18 | acacia Senegal gum (1) |
| POLY 19 | potato starch modified (1) |
| POLY 20 | corn starch modified (1) |
| POLY 21 | sorbitol/sebacic acid copolymer (1) |
| POLY 22 | sodium carboxymethyl starch (1) |
| POLY 23 | carrageenan (1) |
| POLY 24 | carrageenan (2) |
| POLY 25 | hydroxypropyl methyl cellulose (1) |
| POLY 26 | pectin (1) |
| POLY 27 | dehydroxanthan gum (0.5) |
| POLY 28 | dehydroxanthan gum (0.25); hydroxypropyl guar (0.25) |
| POLY 29 | dehydroxanthan gum (0.4); hydroxypropyl guar (0.1) |
| POLY 30 | dehydroxanthan gum (0.5); hydroxypropyl guar (0.5) |
| POLY 31 | dehydroxanthan gum (0.5); hydroxypropyl guar (0.25) |
| POLY 32 | sclerotium gum (1) |
| POLY 33 | xanthan gum (0.25) and carob gum (0.25) |
| POLY 34 | xanthan gum (0.125) and sclerotium gum (0.375) |
| POLY 35 | xanthan gum (0.25) and sclerotium gum (0.75) |
| POLY 36 | xanthan gum (0.1) and sclerotium gum (0.3); hydroxypropyl guar (0.1) |
| POLY 37 | xanthan gum (0.188) and sclerotium gum (0.563); hydroxypropyl guar (0.25) |
| POLY 38 | xanthan gum (0.25) and sclerotium gum (0.75); hydroxypropyl guar (0.5) |
| POLY 39 | xanthan gum (0.125) and sclerotium gum (0.375); hydroxypropyl guar (0.25); |
| | xanthan gum (0.11), sclerotium gum (0.05), lecithin (0.0475), and pullulan |
| | (0.475) |
| POLY 40 | xanthan gum (0.125) and sclerotium gum (0.375); xanthan gum (0.1) |
| POLY 41 | xanthan gum (0.125) and sclerotium gum (0.375); xanthan gum (0.25) |
| POLY 42 | xanthan gum (0.063) and sclerotium gum (0.188); hydroxypropyl guar (0.25) |
| POLY 43 | xanthan gum (0.188) and sclerotium gum (0.563) |
| POLY 44 | xanthan gum (0.125) and sclerotium gum (0.375); hydroxypropyl guar (0.25) |
| POLY 45 | xanthan gum (0.063) and sclerotium gum (0.188); hydroxypropyl guar (0.5) |
| POLY 46 | xanthan gum (0.125) and sclerotium gum (0.375); hydroxypropyl guar (0.5) |
| POLY 47 | xanthan gum (0.25) and sclerotium gum (0.75); hydroxypropyl guar (0.25) |

TABLE 5
Nature Based Polymers Not Tested

| Nature-based polymer | Rationale |
|---|---|
| dilinoleic acid/butanediol copolymer | Film former, no suspending ability |
| dimer dilinoleyl dimer dilinoleate | Binder, no suspending ability |
| algin | Sensitive to electrolytes (incompatible with |
| | salicylic acid + sodium hydroxide) |
| oxidized starch acetate | No suspending ability |
| cyclodextrin | No suspending ability |
| hydroxypropyl guar hydroxypropyltrimonium | Cationic; used for foaming and good |
| chloride | skin/hair feel |
| sodium dextran sulfate | Avoid use of sulfates |
| polyhydroxylstearic acid | For oil-based systems |
| microcrystalline cellulose (and) cellulose gum | MC cellulose used in formula, cellulose gum |
| | loss > storage |
| biosaccharide gum-1 (and) sodium levulinate (and) | Skin conditioning agent, no suspending |
| glyceryl caprylate (and) sodium anisate | ability |
| mannitol (and) cellulose (and) hydroxypropyl | HPMC loss > storage |
| methyl cellulose | |

Progression of Experiments Rationale

In accordance with the disclosure, selection of the at least one nature based polymer may be achieved through a series of tests (“Progression Experiments”) which include:

1. Visual assessment of sample for texture;

2. Visual assessment of sample for separation;

3. Evaluation of gel-sol point, reflected as % crossover value;

4. Viscosity testing;

5. Evaluation of re-gelling in dispenser (which would prevent flow through porous applicator from retainer) the polymer can be dispersed in water and alcohol; the polymer can be dispersed in water and alcohol to form a homogeneous hydroalcoholic dispersion that is free or substantially free of non-dispersed gel bodies; and the polymer must suspend the at least one powder without separation. Embodiments of the cosmetic composition that satisfy all of the test criteria are candidates for selection in porous applicators and are further tested for gel-sol point, viscosity, ability to re-gel. The Examples provided herein demonstrate formulations that fail one or more of the tests and also exemplify inventive compositions that are suitable for maintaining solubility of high salicylic acid in a stable gel dispersion of powders, an suitability for use in a dispensing system comprising a porous applicator with a sponge retainer.

Progression of experiments process:

Test compositions according to the formula in Table 3 are prepared with the various nature-based polymers and subjected to visual evaluation for change in texture, and for separation indicating that the gel cannot retain the powder(s) in suspension.

If test 1 is +, move on to test 2; if test 2 is +, and composition is desired for use in a dispenser that includes a porous applicator, then measure crossover x value, and/or viscosity, and/or re-gelling in the applicator for compatibility with various applicators

TABLE 6
Results with various polymers in the formulation as shown in Table 3 in Tests 1-5.

| | | | | | Test 5 |
|---|---|---|---|---|---|
| | | | | | No re-gelling |
| | | | | | in a porous |
| | Test 1 | Test 2 | Test 3 | Test 4 | applicator, |
| | Visual | No | Gel | Viscosity | preventing |
| | assessment | separation | crossover | (rheomat, M2 | formula flow |
| Polymer | of formula | after | point, | spindle, 24 | through the |
| (from key) | sample | 24 hours | % strain | hours), uD | applicator |
| POLY 1 | + | + | 206.142 | | |
| POLY 2 | + | + | 144.412 | | |
| POLY 3 | + | + | loss > storage | | |
| POLY 4 | + | + | 218.9 | | |
| POLY 5 | + | + | 125.813 | | |
| POLY 6 | + | + | loss = storage | | |
| POLY 7 | + | − | | | |
| POLY 8 | + | − | | | |
| POLY 9 | + | + | loss = storage | | |
| POLY 10 | + | + | 27.6945 (loss ≈ | | |
| | | | storage) | | |
| POLY 11 | + | + | loss > storage | | |
| POLY 12 | − (crumbly | | | | |
| | texture) | | | | |
| POLY 13 | − (crumbly | | | | |
| | texture) | | | | |
| POLY 14 | − (starch has | | | | |
| | to be heated | | | | |
| | to be dispersed) | | | | |
| POLY 15 | + | − | | | |
| POLY 16 | + | − | | | |
| POLY 17 | + | − | | | |
| POLY 18 | + | − | | | |
| POLY 19 | − (starch has | | | | |
| | to be heated | | | | |
| | to be dispersed) | | | | |
| POLY 20 | − (starch has | | | | |
| | to be heated | | | | |
| | to be dispersed) | | | | |
| POLY 21 | − (not water- | | | | |
| | soluble) | | | | |
| POLY 22 | − (starch has | | | | |
| | to be heated | | | | |
| | to be dispersed) | | | | |
| POLY 23 | + | + | loss > storage | | |
| POLY 24 | + | + | 37.2583 (loss ≈ | >M2 spindle | |
| | | | storage) | can measure | |
| POLY 25 | + | + | loss >> storage | | |
| POLY 26 | + | + | loss = storage | | |
| POLY 27 | + | + | 80.44 | 46.7 | |
| POLY 28 | + | + | 159.271 | | |
| POLY 29 | + | + | 104.486 | | |
| POLY 30 | + | + | 164.94 | | |
| POLY 31 | + | + | 160.655 | | |
| POLY 32 | − (gel thickened | | | | |
| | within 1 day) | | | | |
| POLY 33 | + | + | 115.027 | | |
| POLY 34 | + | + | 63.64 | 13.6 | |
| POLY 35 | + | + | 86.0417 | 26.3 | |
| POLY 36 | + | + | 50.14 | 10 | |
| POLY 37 | + | + | 77.7959 | 13 | |
| POLY 38 | + | + | 92.6154 | 83.5 | |
| POLY 39 | + | + | 84.93 | 35.9 | − |
| POLY 40 | + | + | 105.27 | | |
| POLY 41 | + | + | 167.53 | | |
| POLY 42 | + | + | loss > storage | | |
| POLY 43 | + | + | 88.1232 | | |
| POLY 44 | + | + | 42.6677 | 24.7 | + |
| POLY 45 | + | + | loss > storage | | |
| POLY 46 | + | + | 57.93 | 52.6 | |
| POLY 47 | + | + | 91.22 | | |

Below, as set forth in Table 7 and Table 8, specific tested compositional details and results are compiled for certain of the compositions as listed above, and further detail is described herein below.

TABLE 7
Formulations Evaluated in Crossover, Viscosity and Water Absorption Results with Select Compositions

| | | | | | | RATIO OF POLYMERS | |
|---|---|---|---|---|---|---|---|
| FORMULA # | POLYMER 1 | POLYMER 2 | % POLY 1 | % POLY 2 | TOTAL | 1 | 2 |
| POLY 39 | xanthan gum (and) | | 0.5 | | 0.5 | 1.00 | 0.00 |
| | sclerotium gum | | | | | | |
| POLY 46 | xanthan gum (and) | hydroxypropyl | 0.25 | 0.25 | 0.5 | 0.50 | 0.50 |
| | sclerotium gum | guar | | | | | |
| POLY 47 | xanthan gum (and) | | 0.75 | | 0.75 | 1.00 | 0.00 |
| | sclerotium gum | | | | | | |
| INV 1 | xanthan gum (and) | hydroxypropyl | 0.5 | 0.25 | 0.75 | 0.67 | 0.33 |
| | sclerotium gum | guar | | | | | |
| POLY 48 | xanthan gum (and) | hydroxypropyl | 0.25 | 0.5 | 0.75 | 0.33 | 0.67 |
| | sclerotium gum | guar | | | | | |
| POLY 40 | xanthan gum (and) | | 1 | | 1 | 1.00 | 0.00 |
| | sclerotium gum | | | | | | |
| POLY 42 | xanthan gum (and) | hydroxypropyl | 0.75 | 0.25 | 1 | 0.75 | 0.25 |
| | sclerotium gum | guar | | | | | |
| POLY 49 | xanthan gum (and) | hydroxypropyl | 0.5 | 0.5 | 1 | 0.50 | 0.50 |
| | sclerotium gum | guar | | | | | |
| POLY 50 | xanthan gum (and) | hydroxypropyl | 1 | 0.25 | 1.25 | 0.80 | 0.20 |
| | sclerotium gum | guar | | | | | |
| POLY 43 | xanthan gum (and) | hydroxypropyl | 1 | 0.5 | 1.5 | 0.67 | 0.33 |
| | sclerotium gum | guar | | | | | |
| COMP 3 | xanthan gum (and) | hydroxypropyl | 0.75 | 0.25 | 1 | 0.75 | 0.25 |
| | sclerotium gum | guar | | | | | |

TABLE 8
Crossover, Viscosity and Water Absorption Results
with Select Compositions as listed in Table 7

| | | | crossover | | | |
|---|---|---|---|---|---|---|
| | | SUSPEND | x value, % | viscosity | | |
| FORMULA | VISUALLY | POWDER | less than | M2(t10), | other | water |
| # | PASS | PASS | 85% PASS | uD | notes | absorption* |
| POLY 39 | PASS | PASS | 63.64 | 13.6 | | |
| | | | PASS | ~PASS | | |
| POLY 46 | PASS | PASS | 0 | | loss > | |
| | | | FAIL | | storage | |
| POLY 47 | PASS | PASS | 88.1232 | | | |
| | | | ~PASS | | | |
| INV 1 | PASS | PASS | 42.6677 | 24.7 | *values | 15 seconds |
| | | | PASS | PASS | from 1 kg | PASS |
| | | | | | batch | |
| POLY 48 | PASS | PASS | 0 | | loss = | |
| | | | FAIL | | storage | |
| POLY 40 | PASS | PASS | 86.0417 | 26.3 | | |
| | | | ~PASS | PASS | | |
| POLY 42 | PASS | PASS | 89.5154 | | | |
| | | | ~PASS | | | |
| POLY 49 | PASS | PASS | 57.93 | 52.6 | | |
| | | | PASS | ~PASS | | |
| POLY 50 | PASS | PASS | 91.22 | | | |
| | | | ~PASS | | | |
| POLY 43 | PASS | PASS | 92.6154 | 83.5 | | |
| | | | ~PASS | ~PASS | | |
| COMP 3 | PASS | PASS | 65.453 | 31.1 | *values | 1 min/20 |
| | | | PASS | PASS | from 1 kg | min fully |
| | | | | | batch | FAIL |

*Water absorption means time for absorption of 0.05 grams into porous applicator after initially absorbing composition into porous applicator and then 1 week drying.

As described above in Tables 6-8, visual suspension of powders, rheology, viscosity, and gelling in the porous applicator were measurements used to screen natural polymers and combinations of natural polymers for the inventive cosmetic composition.

Samples screened were prepared with 0.5%, perlite, microcrystalline cellulose, alcohol denat, sodium hydroxide to pH adjust, and various natural polymers. Some of the polymers were eliminated without batching or immediately after batching due to incompatibility with the rest of the formula. For example, sorbitol/sebacic acid copolymer is not soluble in water. Gellan gum (tested at 0.25% and 0.5%) was eliminated due to high electrolyte sensitivity. Many of the natural polymers were unable to suspend the powders with 0.5% salicylic acid, resulting in batches that displayed a clear separation into two layers. Examples include cellulose gum (0.5%), pullulan (1%), and hydroxypropyl starch phosphate (0.5%). If no visual separation was observed after 24 hours, the sample was tested for gel crossover point using a rheometer.

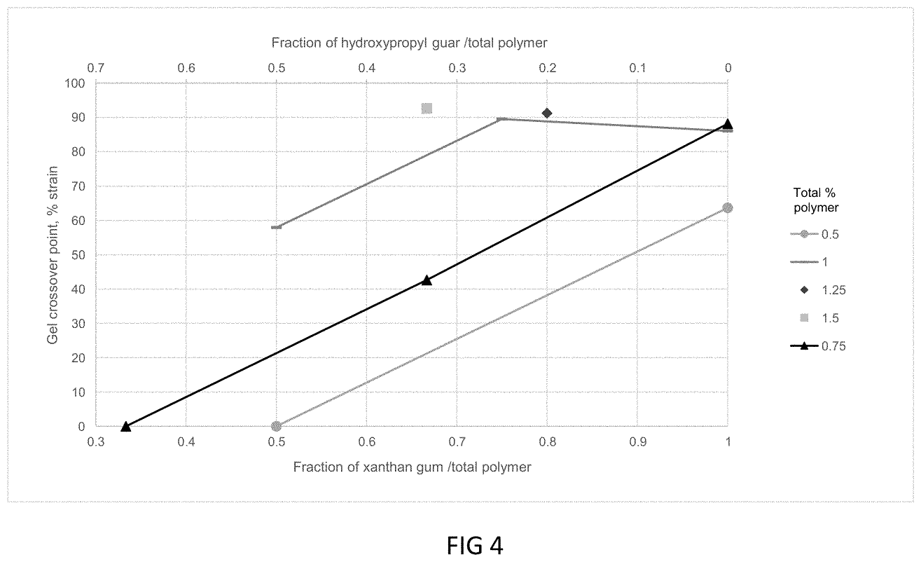

Tables 7 and 8 provide results for a subset of the tested compositions that include inventive and comparative POLY compositions, where the details are provided in the context of total and fractional amounts of nature-based polymers. FIG. 4 provides a graphical representation of the relationship between the polymer fraction and gel crossover point.

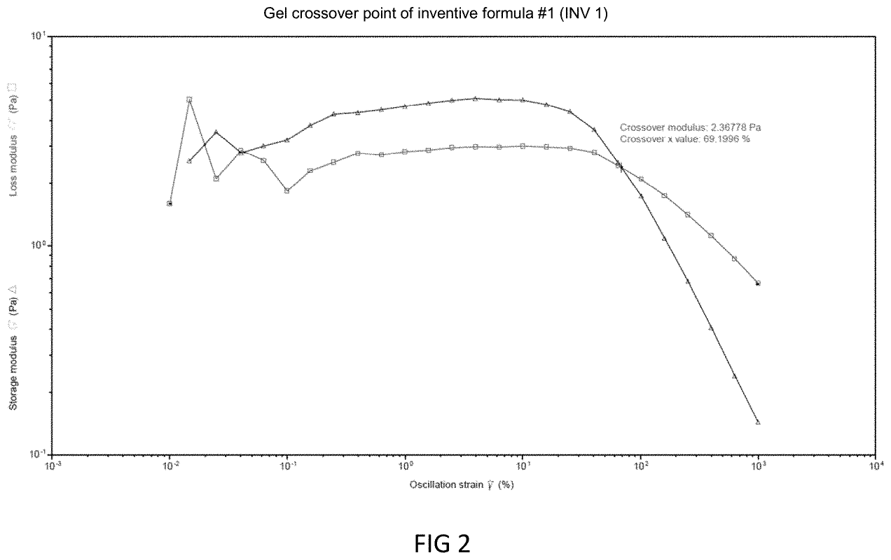

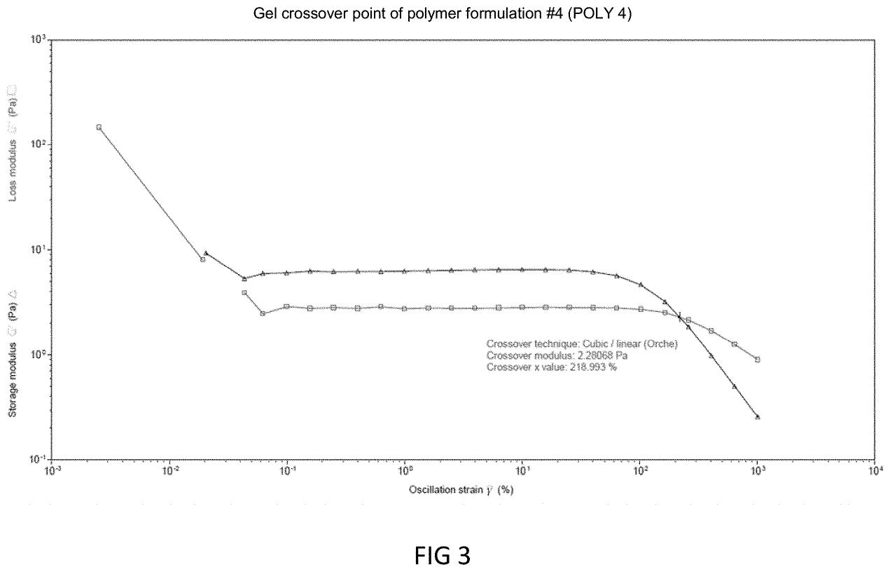

In view of the results obtained with the various inventive compositions, for those composition embodiments that are suitable for use in a porous applicator, the preferred gel crossover point was determined to be between about 35% and about 85% strain to achieve flow through a porous applicator, and gel crossover point can be concentration-dependent for certain polymers. For example, referring now to the results shown in Table 7, various nature-based polymers were evaluated for gel crossover point, and as shown, xanthan gum exhibited gel crossover points of about 219% (POLY 4) and about 126% strain at 0.5% and 0.2%, respectively, and the loss modulus was equal to the storage modulus at 0.1%. Thus, xanthan gum alone could not function as the suspending polymer in this formula at any concentration. If the gel crossover point of the sample was between about 35% and about 85% strain, it was tested for viscosity. Referring to the drawings, FIG. 2 shows a graphical representation of the gel crossover point determination for INV 1 (69.2% strain and a viscosity of 25 uD, determined using an M2 spindle. FIG. 3 shows results with a comparative composition, POLY 4, which demonstrates a crossover point of about 219% strain.

If the viscosity of the sample is too high, it will not flow through the porous applicator. The higher the viscosity of the formula, the lower the gel crossover point must be for proper flow through the porous applicator. The optimum viscosity was determined to be in the range from about 20-30 uD using an M2 spindle on Maple instruments RM180 rheomat. Some polymers provided a gel crossover point in the target range, but the viscosity was too high or too low. For example, carrageenan (21; 2%) provided a gel crossover point of 37% strain, but was too viscous (>80 uD).

As shown above, a few natural polymer combinations were found to be promising, and based on the findings, a number of compositions were tested with the polymers at varying concentrations to achieve a balance of stability, gel crossover point, and viscosity. The last screening test was to ensure that the bulk formula would not re-gel and dry in the porous applicator, which would prevent passage through the applicator.

Sample formulas were absorbed into the porous applicator overnight, removed, and allowed to dry overnight. A drop of water, approximately 0.05 g, was placed on the applicator and the time for the water to absorb into the applicator was measured. If the sample re-gelled in the applicator, water would not be able to pass through. This was the case for COMP 3, which had 0.5% sclerotium gum (and) xanthan gum, 0.25% xanthan gum (and) sclerotium gum (and) lecithin (and) pullulan, and 0.25% hydroxypropyl guar. Referring again to the above tables, COMP 3 was able to suspend the powders, had a gel crossover point of 65% strain, and a viscosity of 31.1 uD. However, it took about 1 minute for the water to absorb most of the way into the soaked porous applicator, and 20 minutes to fully absorb. The presence of powders that had passed through the porous applicator were detected using plane- and cross-polarized microscopy. And, the applicators were also evaluated for retention of sol-gel crossover point stability by evaluating the time taken for dried applicators to absorb water, wherein when water was fully absorbed into a soaked applicator within 15 seconds then the inventive formula has been deemed to not re-gel in the applicator.

Long term stability of the invention was measured by visual and odor assessment, pH, and viscosity over a two-month period at the following temperatures: 4, 25, 37, and 45 C, as well as a 10-day freeze/thaw cycle. Unstable prototypes typically exhibited a clear separation into two distinct layers or a significant change in viscosity. This invention composition INV 1 is stable over time with 0.5% salicylic acid, 0.5% sclerotium gum (and) xanthan gum, and 0.25% hydroxypropyl guar.

Referring again to the above tables, INV 1 is stable with 0.5% salicylic acid, has a gel crossover point of 69.2% strain and a viscosity of 25 uD using an M2 spindle, allowing it to pass through a porous applicator. A drop of water could absorb into an applicator soaked in this formula within 15 seconds, indicating that this formula does not re-gel in a porous applicator. This formula is 99.11% natural. Referring again to the above tables, COMP 3 is stable with 0.5% salicylic acid, has a gel crossover point of 65.5% strain and a viscosity of 31 uD using an M2 spindle, allowing it to pass through a porous applicator. However, a drop of water required up to 20 minutes to absorb into an applicator soaked in this formula, indicating that this formula re-gels in a porous applicator, blocking passage through the applicator.

### Example 5. Evaluation of Sweat Resistance of Inventive and Comparative Compositions

TABLE 8
Sweat resistance

| | Sweat Resistance - Performance Index Compared to Positive and Negative References | | | | | |
|---|---|---|---|---|---|---|
| New Formula | Pre-Spray | | 6 Min Post-Spray | | 12 Min Post-Spray | |
| Number | Average | Error | Average | Error | Average | Error |
| INV 1 | 30.55 | 5.54 | 22.08 | 7.53 | 8.90 | 11.03 |
| COMP 4 | 16.72 | 6.41 | 5.84 | 5.75 | −12.97 | 11.87 |
| INV 2 | 64.68 | 8.80 | 62.49 | 6.55 | 55.66 | 10.41 |
| INV 3 | 55.04 | 4.11 | 51.01 | 3.09 | 41.94 | 5.49 |
| INV 4 | 50.93 | 2.72 | 45.77 | 1.53 | 35.03 | 5.94 |
| INV 5 | 40.72 | 1.68 | 35.23 | 3.98 | 23.00 | 9.32 |
| INV 6 | 55.71 | 2.64 | 51.60 | 1.20 | 47.65 | 3.98 |
| INV 7 | 47.23 | 5.69 | 42.42 | 3.82 | 32.79 | 5.98 |
| NEG CTRL | 0 | 0 | 0 | 0 | 0 | 0 |
| POS CTRL | 100 | 0 | 100 | 0 | 100 | 0 |

| | |
|---|---|
| performance index | indication |
| >=100 | better than positive reference |
| 0-100 | better than negative reference and worse than positive reference |
| <=0 | worse than negative reference |

Negative Control Test Composition for Sweat Study
COMPOSITION FORMULA NEGATIVE CONTROL
(NO/LOW SWEAT RESISTANCE; Normaderm placebo)

| | w/w % |
|---|---|
| RM INCI | formula |
| WATER | 67.85 |
| GLYCERIN | 7 |
| PROPYLENE GLYCOL | 2 |
| AMMONIUM POLYACRYLOYLDIMETHYL TAURATE | 1 |
| DIMETHICONE | 7 |
| PEG-12 DIMETHICONE | 0.7 |
| DIMETHICONE | 4 |
| BIOSACCHARIDE GUM-1 | 2 |
| CAPRYLYL GLYCOL | 0.4 |
| MENTHOL | 0.05 |
| ALCOHOL DENAT. | 8 |

Positive Control Test Composition for Sweat Study
COMPOSITION FORMULA POSITIVE CONTROL
(COMPLETE SWEAT RESISTANCE; Normaderm placebo)

| | |
|---|---|
| WATER | 65.85 |
| GLYCERIN | 7 |
| PROPYLENE GLYCOL | 2 |
| AMMONIUM POLYACRYLOYLDIMETHYL TAURATE | 1 |
| DIMETHICONE | 7 |
| PEG-12 DIMETHICONE | 0.7 |
| DIMETHICONE | 4 |
| BIOSACCHARIDE GUM-1 | 2 |
| SILICA SILYLATE | 2 |
| CAPRYLYL GLYCOL | 0.4 |
| MENTHOL | 0.05 |
| ALCOHOL DENAT. | 8 |

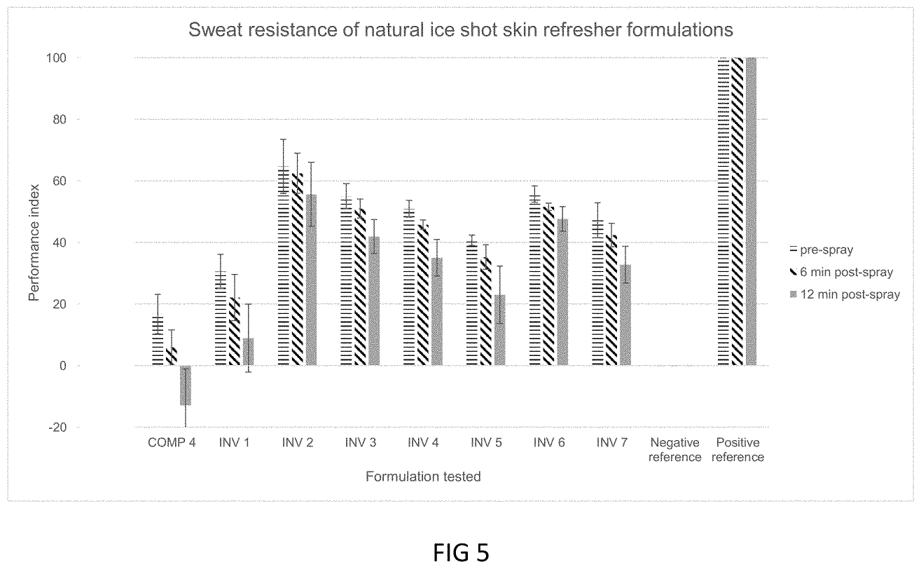

Sweat resistance was assessed in vitro using a Normaderm placebo with no powders as the negative reference and a Normaderm formula with 2% silica silylate as a positive reference, having the composition formulae as listed above. Three replicates were tested for each formulation, and the data shown sets forth the averages and errors obtained. The sweat resistance data are shown above in Table 8 and in FIG. 5. Drawdowns of various prototypes and the references were sprayed with artificial sweat (Oleic acid, water, oleth-10) and measured with a glossmeter. Additional natural powders to be covered by this patent application can be added to the formulation of INV 1 to boost sweat resistance performance such as cellulose or kaolin.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” as used herein, means that there may be one, two, three or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the cosmetic composition, typically 0% by weight, based on the total weight of the cosmetic composition. The term “essentially free” means that, while it prefers that no excluded material is present in the cosmetic composition, it is possible to have very small amounts of the excluded material in the cosmetic composition of the invention, provided that these amounts do not materially affect the advantageous properties of the cosmetic composition. In particular, “essentially free” means that excluded material can be present in the cosmetic composition at an amount of less than about 0.1% by weight, based on the total weight of the cosmetic composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. As used herein a range of ratios is meant to include every specific ratio within, and combination of subranges between the given ranges.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition, comprising:
a hydroalcoholic gel dispersion, comprising a blend of
(i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers,
(ii) at least one C2-C5 alcohol,
(iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and
(iv) a blend of at least two powders, at least one powder selected from inorganic powders and organic powders, and at least one nature-based organic powder,
wherein the at least one beta hydroxy acid is dissolved in the hydroalcoholic gel dispersion and the blend of at least two powders is dispersed in the hydroalcoholic gel dispersion.

2. The cosmetic composition according to claim 1, wherein the at least one beta hydroxy acid is salicylic acid.

3. The cosmetic composition according to claim 1, wherein at least one beta hydroxy acid comprises salicylic acid present in the composition in the range from about 0.2% to about 2%, by weight, based on the total weight of the composition.

4. The cosmetic composition according to claim 1, wherein the at least one C2-C5 alcohol is selected from ethanol, propanol, butanol, pentanol, isopropanol, isobutanol and isopentanol.

5. The cosmetic composition according to claim 1, wherein the at least one C2-C5 alcohol comprises denatured alcohol present in the composition in the range from about 5% to about 20%, by weight, based on the total weight of the composition.

6. The cosmetic composition according to claim 1, wherein the pH of the cosmetic composition is in the range from about 3.9 to about 4.5.

7. The cosmetic composition according to claim 1, wherein the blend of powders comprises perlite, and one or more of cellulose, microcrystalline cellulose and kaolin.

8. The cosmetic composition according to claim 1, wherein the cosmetic composition excludes one or more powders comprising Nylon-12, polymethyl methacrylate, polyethylene beads, and powders which are considered microplastics.

9. The cosmetic composition according to claim 1, wherein the at least one inorganic powder is present in the cosmetic composition in an amount that ranges from about 0.5% to about 1% by weight, and each one of the at least one organic powder is present in the cosmetic composition in an amount that ranges from about 0.25% to about 2% by weight, by weight based on the weight of the composition.

10. The cosmetic composition according to claim 1, wherein the at least one nature-based polymer further includes one or more polymers selected from acacia Senegal gum, Caesalpinia spinosa gum, agar, carob gum, carrageenan, carrageenan (lambda, iota kappa), cellulose gum, corn starch modified, dehydroxanthan gum, gellan gum, guar gum, guar hydroxypropyltrimonium chloride, gum acacia, gum arabic, gum ghatti, gum karaya, gum tragacanth, hydroxypropyl guar, hydroxypropyl methyl cellulose, lecithin, pectin, potato starch modified, pullulan, sodium carboxymethyl starch, sorbitol/sebacic acid copolymer, and mixtures thereof.

11. The cosmetic composition according to claim 1, wherein each of the at least one nature-based polymer is present in an amount that ranges from about 0.2% to about 0.6% by weight.

12. The cosmetic composition according to claim 1, wherein the cosmetic composition excludes synthetic polymers selected from Ammonium Acryloyldimethyltaurate/Steareth-25 Methacrylate Crosspolymer, polyacrylate crosspolymer 6 and an acrylates/C10-30 Alkyl acrylate crosspolymer.

13. The cosmetic composition according to claim 1, wherein the cosmetic composition excludes polymers selected from dilinoleic acid/butanediol copolymer, dimer dilinoleyl dimer dilinoleate, algin, oxidized starch acetate, cyclodextrin, hydroxypropyl guar hydroxypropyltrimonium chloride, sodium dextran sulfate, polyhydroxylstearic acid, microcrystalline cellulose (and) cellulose gum, biosaccharide gum-1 (and) sodium levulinate (and) glyceryl caprylate (and) sodium anisate, and mannitol (and) cellulose (and) hydroxypropyl methyl cellulose.

14. The cosmetic composition according to claim 1, wherein the cosmetic composition has a gel crossover point ranging from about 35% to about 85% strain.

15. The cosmetic composition according to claim 1, wherein the cosmetic composition has a gel crossover point which allows it to suspend the at least one powder and pass through a porous applicator with shear.

16. A cosmetic composition, comprising: (a) salicylic acid present in an amount in the range from about 0.2% to about 2%, and an (b) aqueous hydroalcoholic gel dispersion, comprising: (i) denatured alcohol in an amount that is about 20% by weight, (ii) perlite present in the cosmetic composition in an amount that ranges from about 0.5% to about 1% by weight, microcrystalline cellulose present in the cosmetic composition in an amount that ranges from about 1.4% to about 2% by weight, and one or more of cellulose and kaolin, each present in the cosmetic composition in an amount that ranges from about 0.25% to about 1% by weight, and (iii) at least one of a blend of xanthan gum and sclerotium gum and hydroxypropyl guar, the blend of xanthan gum and sclerotium gum, when present, is present in an amount that ranges from about 0.25% to about 0.75% by weight, and the hydroxypropyl guar, when present, is present in an amount that ranges from about 0.1% to about 0.4% by weight.

17. A cosmetic dispensing system, comprising:
a cosmetic composition that has a gel crossover point suitable for dispensing through a porous applicator, the cosmetic composition comprising:
a hydroalcoholic gel dispersion, comprising a blend of
(i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers,
(ii) at least one C2-C5 alcohol,
(iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and
(iv) a blend of at least two powders, at least one powder selected from inorganic powders and organic powders, and at least one nature-based organic powder,
wherein the at least one beta hydroxy acid is dissolved in the hydroalcoholic gel and the blend of at least two powders is dispersed in the hydroalcoholic gel; and
a dispenser that includes a container that includes a retainer and an absorbent component disposed within the retainer, and a porous applicator that is in contact on a first side with the absorbent component in the retainer, and is exposed on a second side for contacting keratinous tissue to be treated,
wherein the cosmetic composition is absorbed within the absorbent component and retained in the retainer, which retains the cosmetic composition in a dispersed gel state until dispensed; and
wherein the dispenser optionally comprises a cap for covering the porous applicator when the dispensing system is not in use.

18. The cosmetic dispensing system according to claim 17, wherein the porous applicator of the cosmetic dispensing system includes one or more of:
a. on at least the exposed surface intended for contacting skin, a plurality of pores having an average size in a range from about 30 to about 200 microns;
b. on at least the first surface intended for contacting the sponge containing the cosmetic composition in the retainer, a plurality of pores having an average size in a range from about 90 to 200 microns;
c. from the first surface to the second surface throughout the porous applicator, a plurality of pores having an average size in a range from 1 to about 1000 microns;
d. the porosity through the porous material from the first surface to the second surface is uniform and continuous;
e. the porosity through the porous material from the first surface to the second surface is variable from larger pores at the first surface to smaller pores at the second surface; and
f. the porosity through the porous material from the first surface to the second surface is variable from smaller pores at the first surface to larger pores at the second surface.

19. A method for enhancing the appearance of acne prone skin, comprising:
providing a dispensing system comprising: a cosmetic composition that has a gel crossover point suitable for dispensing through a porous applicator, and a dispenser that includes a container that includes a retainer and an absorbent component disposed within the retainer, and a porous applicator that is in contact on a first side with the absorbent component in the retainer, and is exposed on a second side for contacting keratinous tissue to be treated,
wherein the cosmetic composition comprises:
a hydroalcoholic gel dispersion, comprising a blend of
(i) at least one nature-based polymer that includes sclerotium gum (and) xanthan gum, and hydroxypropyl guar, the at least one nature-based polymer optionally further including one or more nature-based polymers selected from polysaccharides, proteins, and bacterial polyesters, the at least one nature-based polymer excludes synthetic organic polymers,
(ii) at least one C2-C5 alcohol,
(iii) at least one beta hydroxy acid selected from -n-octanoylsalicylic acid, salicylate, sodium salicylate, willow extract, beta hydroxybutanoic acid, tropic acid, and trethocanic acid, the beta hydroxy acid present in the cosmetic composition in an amount that is up to about 2.0%; and
(iv) a blend of at least two powders, at least one powder selected from inorganic powders and organic powders, and at least one nature-based organic powder,
wherein the at least one beta hydroxy acid is dissolved in the hydroalcoholic gel dispersion and the blend of at least two powders is dispersed in the hydroalcoholic gel dispersion; and
wherein the dispenser containing the cosmetic composition is used to apply the composition to skin that is prone to acne by utilizing the dispenser to apply the cosmetic composition by contacting the porous applicator to skin using a dabbing motion to dispense a light coating of the cosmetic composition onto select areas of the skin in dispensed doses that are in an amount that is from about 0.01 mg to about 100 mg per application stroke.
